(19) United States
(12) Patent Application Publication
(10) Pub. No.: US 2010/0278913 A1
(43) Pub. Date: Nov. 4, 2010

(54) CHEWABLE TABLET

(75) Inventors: Frederick D. Sancilio, Jupiter, FL (US); Frank C. Greaves, Weston, FL (US)

Correspondence Address: STROOCK & STROOCK & LAVAN LLP, 180 MAIDEN LANE, NEW YORK, NY, 10038, US

(73) Assignee: Sancilio & Company

(21) Appl. No.: 12/433,096

(22) Filed: Apr. 30, 2009

(51) Int. Cl. A61K 9/20; A61K 47/26

(52) U.S. Cl. 424/465; 514/781

(57) ABSTRACT

An excipient base for a chewable tablet comprising xylitol, sucralose and microcrystalline cellulose. The excipient base provides for an improved taste, stabilization, and mouthfeel qualities for the chewable tablet, as well as a greater likelihood for dosage compliance and use. The excipient base also enables the tablet to be directly compressible, free flowing and non-tacky, thus avoiding wet granulation techniques that can degrade the product and add to the cost of the manufacturing process. The excipient base also allows for the production of a chewable tablet that is suitable for administration to persons and animals having diabetes or hypoglycemia, and does not promote tooth decay or dental caries.

### FIELD OF THE INVENTION

The present invention is directed at a chewable pharmaceutical dosage form. More particularly, the present invention is directed to a chewable tablet for administering multivitamin and/or mineral blends, a pharmaceutical agent, medicament or other active ingredient by employing an excipient base that has an improved taste and mouthfeel qualities. Further, the present invention is directed to a chewable tablet that is suitable for administration to persons or animals suffering from diabetes and hypoglycemia. Further, the present invention is directed to a chewable tablet that does not promote tooth decay or dental caries.

### BACKGROUND OF THE INVENTION

Pharmaceutical and nutritional supplement dosage forms intended for oral administration are typically provided in solid form as tablets, capsules, pills, lozenges, or granules. The tablet form is swallowed whole, chewed in the mouth, or dissolved sublingually. Absorption of the active moiety depends upon its release from the dosage form and may be controlled by several different technologies.

Chewable systems are often employed in the administration of pharmaceuticals and nutritional supplements where it is impractical to provide a tablet for swallowing whole. Further, the act of chewing helps to break up the tablet particles as the tablet disintegrates and may increase the rate of absorption by the digestive tract. Chewable systems are also advantageous where it is desirable to make an active ingredient available topically in the mouth or throat for both local effects or systemic absorption. Chewable dosage forms are also utilized to improve drug administration in pediatric and geriatric patients.

In addition to the therapeutic ingredients of pharmaceutical tablets as well as vitamins and/or minerals (commonly referred to, and referred to here, as “actives”), materials inert and non-reactive with respect to the actives (commonly referred to, and referred to here, as “excipients”) are added to the tablet formulation to confer specific properties not related to the activity of the therapeutic agents. In many cases, an active substance (such as aspirin) may not be easily administered and absorbed by the human body; in such cases the substance in question may be dissolved into or mixed with an excipient. Excipients are also sometimes used to bulk up formulations with very potent active ingredients to allow for convenient and accurate dosage. In addition to their use in the single-dosage quantity, excipients can be used in the manufacturing process to aid in the handling of the active substance concerned. Depending on the route of administration and form of medication, different excipients may be used.

Often, once an active ingredient has been purified, it cannot stay in purified form for long. In many cases it will denature, fall out of solution or stick to the sides of the container. To stabilize the active ingredient, excipients can be added, ensuring that the active ingredient stays “active” and stable for a sufficient period of time.

Excipients such as diluents, binders, glidants, and lubricants are also often added as processing aids to make the tableting operation more effective. Other excipients such as microcrystalline cellulose are often added to improve the compression of the tablets. Still other types of excipients often added to pharmaceutical tablets and nutritional supplement tablets are those that enhance or retard the rate of disintegration of the tablet in the patient, improve the taste of the tablet or impart a color to the tablets. In some instances, excipients will provide more than one of these benefits to the finished tablet. Although excipients are classified as inert materials, they are only inert in the sense that they are not actives and do not provide a therapeutic effect in themselves, but they make delivery of the therapeutic agent in the most effective manner possible.

Chewable tablets, regardless of their geometry, represent a particular form of oral dosage; they are intended to be chewed in the mouth by the patient and are not intended to be swallowed intact. Many chewable formulations are intended to be used to provide a known dosage of active to children or people who either will refuse to swallow an intact tablet or have difficulty doing so. Such tablets are often used to administer analgesics, antacids, antibiotics, anticonvulsants, vitamins, multivitamins, minerals, multivitamin and mineral blends, and laxatives, by way of example. Because vitamins are generally unstable, however, finding a good tasting formula that is also stable has been difficult.

In addition to the foregoing, chewable tablets have several advantages that make them the method of choice in delivering certain types of therapeutic agents or actives to an even greater population. One such advantage is that certain types of tablets, because of the large size of the dosage, must be unusually large and, therefore, difficult to swallow. In some cases the effectiveness of the therapeutic agent is improved by the reduction in size that occurs during mastication of the tablets before swallowing. Furthermore, patient compliance with the prescribed therapy, such as antacid treatment, is enhanced by the use of smaller, more convenient tablets that may be consumed when it may be inconvenient to swallow pills. This is particularly true when the therapy would involve, for example, a liquid suspension of the therapeutic agent that would be inconvenient to transport.

Excipients when added to chewable tablets must not only be inert in respect of the active, but also preferably provide pleasant mouthfeel and/or prevent toothpacking, grittiness, and the like, without imparting any unpleasant characteristics to the tablets as they are chewed. As described in U.S. Pat. No. 5,686,107, which is incorporated herein by reference, microcrystalline cellulose in various forms, such as products of the Pharmaceutical and Biosciences Division of FMC Corporation, Philadelphia, Pa. U.S.A. sold under the Avicel® brand, are frequently used as excipients in pharmaceutical tablets, but have not found ready acceptance in chewable tablet applications because of the astringent mouthfeel they may impart to a tablet as it is being chewed.

Generally, chewable tablets are made by direct compression of a mixture of tabulating compounds including the active ingredient, flavoring, binders, and other recipients. As described in U.S. Pat. No. 5,637,313, which is incorporated herein by reference, the mixture is fed into a die cavity of a tablet press and a tablet is formed by applying pressure. Hardness of the resulting tablet is a direct function of the compression pressure employed and excipients used. A softer tablet, having an easier bite-through, may be prepared by adding a disintegrant, such as alginic acid, to the tablet mix. Alternatively, a softer tablet may be formed by employing reduced compression pressures. In either case, the resulting tablet is often softer, fragile, brittle, and easily chipped.

A wet granulation technique for forming tablets containing microcrystalline cellulose derived from an aqueous slurry is described in U.S. Pat. No. 6,936,277, which is incorporated herein by reference. As described therein, the tablet can be formed by pressure being applied to the material to be tableted on a tablet press. A tablet press includes a lower punch that fits into a die from the bottom and a upper punch having a corresponding shape and dimension that enters the die cavity from the top after the tableting material fills the die cavity. The tablet is formed by pressure applied on the lower and upper punches. The ability of the material to flow freely into the die is important in order to insure that there is a uniform filling of the die and a continuous movement of the material from the source of the material, e.g. a feeder hopper. The lubricity of the material is crucial in the preparation of the solid dosage forms since the compressed material must be readily ejected from the punch faces.

Palatability and mouthfeel are important characteristics to be considered in providing a chewable dosage form for an active pharmaceutical medicament or nutritional supplement. Unfortunately, many pharmaceuticals and other active ingredients have a bitter or otherwise unpalatable taste, or an unacceptable mouthfeel, due to the grittiness or chalkiness of the compound, or both. These characteristics make it difficult to prepare acceptable dosage forms using the current state of the art for chewable dosage forms, since objectionable taste and/or mouthfeel continues to be a problem and make it less likely to obtain dosage compliance by the user. These problems can be more prevalent when considering the natural poor taste of vitamins and minerals, as well as the bitter taste and poor mouth feel of sodium fluoride.

As a result, many approaches have been tried in attempting to overcome these problems. The poor taste of a pharmaceutical or other active ingredient may be masked by using various flavoring compounds, and/or sweeteners. Encapsulation of the active ingredient may also serve to mask bitterness and other undesirable tastes. These approaches do not effect the physical state of the dosage from currently employed in the art. For example, chewable vitamin tablets are typically prepared as a compressed tablet, incorporating one or more active ingredients (e.g., vitamins), a sweetener and flavoring agent to mask the taste of the active ingredient, and a binder, typically microcrystalline cellulose.

Attempts have been made to reduce the grittiness and/or chalkiness of the compressed tablet by coating particles of the active ingredient with oils or fats prior to incorporation into the delivery system. See U.S. Pat. Nos. 4,327,076, 4,609,543 and 5,686,107, which are incorporated herein by reference. In this way, the grittiness or chalkiness of the particles in the mouth is reduced. After swallowing, the oil or fat is digested and the drug particle can dissolve in the gastric contents. But the addition of particles coated with fats or oils to the tablet mix can decrease the binding of the tableting ingredients and cause a reduction in the tablet hardness.

Other techniques for providing a chewable delivery system involve the use of a gum base. Gum bases are insoluble elastomers that form the essential element for chewing gum. The gum base is typically blended with one or more sweeteners to obtain a confectionery gum. A coating containing the active ingredient is then applied over the confectionery gum. As the dosage form is chewed, the coating fractures and/or dissolves in the mouth and is swallowed.

Despite these disclosures there is an ongoing need for a chewable tablet delivery system, particularly for children, that is pleasant tasting and provides improved mouthfeel qualities. There is also an ongoing need for a chewable tablet delivery system that is pleasant tasting, has acceptable mouthfeel, and that is suitable for administration to persons and animals having diabetes or hypoglycemia, and does not promote tooth decay or dental caries.

### SUMMARY OF THE INVENTION

The present invention relates to a novel chewable tablet with improved taste and mouthfeel properties. More particularly, the present invention relates to a novel excipient base for a chewable tablet comprising xylitol, sucralose and microcrystalline cellulose. The novel excipient base provides for an improved taste, stabilization, and mouthfeel qualities for the chewable tablet. In a preferred embodiment, the excipient base of the present invention comprises, by weight of the tablet about 40-70% xylitol, about 15-20% microcrystalline cellulose, and about 0.01-1% sucralose. The novel excipient base of the present invention also enables the tablet to be directly compressible, free flowing and non-tacky, thus avoiding wet granulation techniques that can degrade the product and add to the cost of the manufacturing process. The novel excipient base of the present invention also provides for a chewable tablet that is acceptable for administration to persons and animals having diabetes and hypoglycemia, and a chewable tablet that does not promote tooth decay or dental caries.

Other objects, features, and characteristics of the present invention will become more apparent upon consideration of the following detailed description which forms a part of this specification.

### BRIEF DESCRIPTION OF THE DRAWINGS

The particular features and advantages of the invention as well as other objects will become apparent from the following description, taken in connection with the accompanying drawings, in which:

FIG. 1 is a process flow diagram.

### DETAILED DESCRIPTION OF A PREFERRED EMBODIMENT

Other than in the operating examples, or where otherwise indicated, all numbers expressing quantities of ingredients, parameters, or reaction conditions used herein are to be understood as modified in all instances by the term “about”. Unless indicated otherwise, all percentages recited in the specification, including the claims, are weight percentages.

The present invention is directed towards novel formulations for chewable pharmaceutical, nutritional supplement and other tablets that have improved taste, stabilization, and mouthfeel. In addition, the present invention is directed towards a tablet that can be directly compressed, is free flowing and non-tacky, thereby avoiding wet granulation techniques that can degrade the product and add to the cost of the manufacturing process.

In a first embodiment, the present invention relates a novel excipient base for a chewable tablet, the excipient base comprising xylitol, sucralose and microcrystalline cellulose. In a preferred embodiment, the excipient base comprises generally, by total weight of the tablet, 40-70% xylitol, 15-20% microcrystalline cellulose, and 0.1-0.2% sucralose, more preferably 60% xylitol, 18% microcrystalline cellulose and 0.15% sucralose, and most preferably, 65% xylitol, 20% microcrystalline cellulose, and 0.2% sucralose.

Xylitol has many beneficial properties over sugar and other sweeteners. Xylitol is an organic compound with the formula (CHOH)₃(CH₂OH)₂ and is roughly as sweet as sucrose with only two-thirds the food energy. One teaspoon (5 mL) of xylitol contains 9.6 calories, as compared to one teaspoon of sugar, which has 15 calories. Xylitol has virtually no aftertaste, and is advertised as “safe for diabetics and individuals with hypoglycemia.” This is because sugar-alcohols have less impact on a person's blood sugar than regular sugars. Xylitol is a “tooth friendly” sugar substitute. In addition to not encouraging tooth decay (by replacing dietary sugars), xylitol may actively aid in repairing minor cavities caused by dental caries. Research confirms a plaque-reducing effect and suggests that the compound, having some chemical properties similar to sucrose, attracts and then “starves” harmful micro-organisms, allowing the mouth to remineralise damaged teeth with less interruption.

Sucralose is approximately 600 times sweeter than sugar, derived from a natural product and does not have effect on insulin.

Examples of Sucralose for use in the excipient base of the present invention include Tate and Lyle, Splenda Sucralose, Micronized NF, Granular NF (DFF-1), or mixtures thereof, of which sucralose granular is the most preferred. Sucralose granular is manufactured by McNeil Nutritionals, LLC, and Tate & Lyle, and is preferred for tableting due in part to its improved handling and flow characteristics, its reduced tendency to form lumps when exposed to moisture, and its easier dispersion and dispensing. Sucralose is also preferred due to its inherent beneficial properties to diabetics when compared to other sweeteners such as mannitol, compressible sugar (sucrose) or fructose. Since Sucralose is not a carbohydrate as the above mentioned sugars, it is a beneficial aid for diabetics and persons with hypoglycemia who must monitor carbohydrate consumption for blood glucose levels.

Microcrystalline cellulose USP is a well known excipient by those familiar in the art. It imparts compressibility to tablet blends and aids the disintegration of the tablet. Examples of microcrystalline cellulose USP that can be used in the excipient base of the present invention include FMC Avicel PH 102, FMC Avicel PH 105, or mixtures thereof, of which PH 105 is the most preferred.

In a second embodiment, the chewable tablet of the present invention comprises an excipient base comprising xylitol, sucralose and microcrystalline cellulose, together with a percentile range of one or more vitamins. A preferred embodiment range of one or more vitamins comprise 0.0005-12% of the total weight of the tablet.

Preferred examples of the vitamins for use in the chewable tablet of the present invention include vitamins A, C, D3, E, B1, B2, Niacin, B6, Folate and B12, or any combinations thereof.

In a third embodiment, the chewable tablet of the present invention comprises an excipient base comprising xylitol, sucralose and microcrystalline cellulose, together with a percentile range of one or more minerals. A preferred embodiment range of one or more minerals comprise of 0.11-9% of the total weight of the tablet. Preferred examples of the minerals for use in the chewable tablet of the present invention include magnesium, phosphorous, copper, sodium fluoride and iron, or any combinations thereof.

In a fourth embodiment, the chewable tablet of the present invention comprises an excipient base comprising xylitol, sucralose and microcrystalline cellulose, together with, by total weight of the tablet, 3-4% magnesium, 4-5% phosphate, and 0.1-0.2% copper (not exceeding 2 mg).

In a fifth embodiment, the chewable tablet of the present invention comprises an excipient base comprising xylitol, sucralose and microcrystalline cellulose, together with a percentile range of one or more active nutrients or food ingredients. In a preferred embodiment, the one or more active nutrients or food ingredients comprise 10-50%, more preferably 20%, and most preferably, 30%, of the total weight of the tablet.

Preferred examples of the active nutrients or food ingredients for use in the chewable tablet of the present invention include protein (amino acids), lipids, carbohydrates, or any combinations thereof.

In a sixth embodiment, the chewable tablet of the present invention comprises an excipient base comprising xylitol, sucralose and microcrystalline cellulose, together with a percentile range of a pharmaceutical active ingredient. In a preferred embodiment, the pharmaceutical active ingredient comprises 15% more preferably 10% and most preferably, 8% of the total weight of the tablet.

Preferred examples of the pharmaceutical active ingredient for use in the chewable tablet of the present invention include analgesics, antacids and laxatives.

In a seventh embodiment, the chewable tablet of the present invention comprises an excipient base comprising xylitol, sucralose and microcrystalline cellulose, together with a percentile range of one or more dietary supplement ingredients. In a preferred embodiment, the one or more dietary supplement ingredients comprise Sodium Fluoride, 0.7%, more preferably 0.1%, and most preferably, 0.3%, (not exceeding 2 mg. of Fluoride) of the total weight of the tablet.

Preferred examples of the one or more dietary supplement ingredients for use in the chewable tablet of the present invention include sodium fluoride and elemental iron or any combinations thereof.

In an eighth embodiment, the chewable tablet of the present invention comprises an excipient base comprising xylitol, sucralose and microcrystalline cellulose, together with one or more minerals comprising no more than 9% by weight of the total tablet, together with (a) one or more vitamins, (b) one or more active nutrients or food ingredients, (c) a pharmaceutical active ingredient, or (d) one or more dietary supplements, wherein (a)-(d) individually or in combination represent 0.07-15% of the total weight of the tablet.

Each of the ingredients used in each of the embodiments described above is free flowing and non-tacky, thus enabling the tablets to be formed by direct compression in a manner known in the art while at the same time avoiding the costs and other production issues present in wet granulation techniques.

The novel excipient base of the present invention provides chewable tablets with improved properties, including improved taste and mouthfeel. The improved properties are detailed further by reference to the following examples, in which various combinations and amounts of various ingredients were prepared and then subject to an internal panel to assess the taste and mouthfeel of the products. The examples described below are not intended to be limiting, but rather as exemplary of the novel excipient base and other tablet formulations of the present invention.

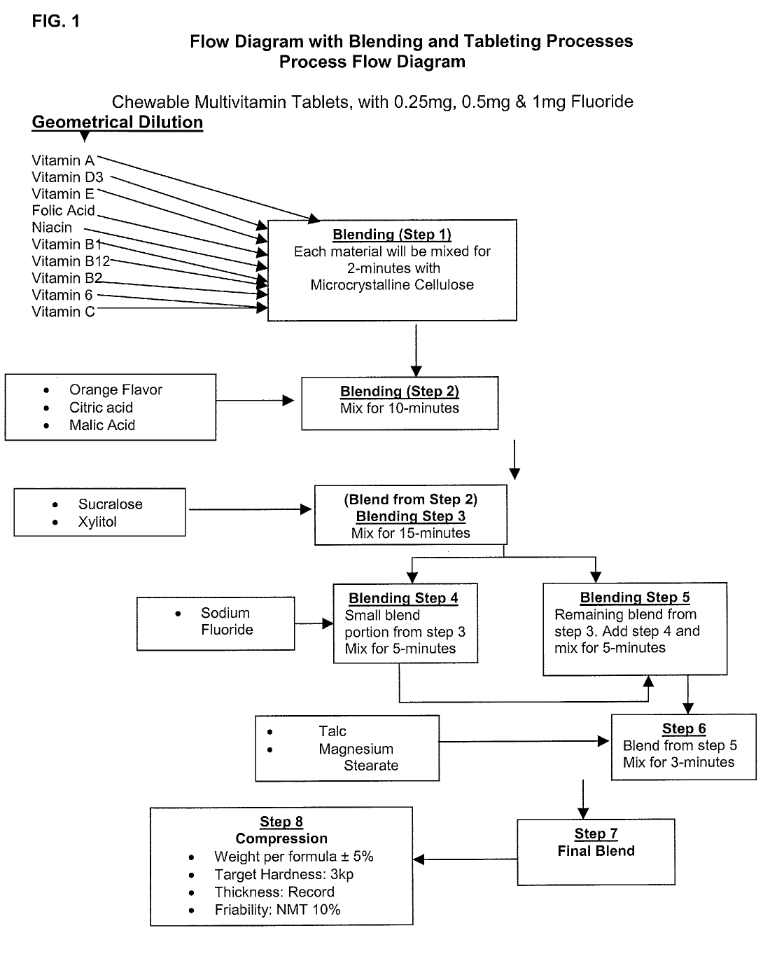

In each of the examples described below, all ingredients were processed, blended and tableted, as shown in the flow diagram in FIG. 1. All tablets had good compressibility, adequate hardness and friability as is appropriate for chewable tablet.

### Blending and Tableting Processes

Step 1. Add each vitamin and/or minerals with the MCC in a 3D blender and mix each for 2 minutes.

Step 2. Add flavor, citric and malic acid to the blender, step 1, and mix an additionally for 10 minutes.

Step 3. The above blend from step 2, were then blended with the Sucrose and Xylitol for 15 minutes.

Step 4. A small portion of the blend from step 3, (referred to from hereon as the Premix), was then blended with the fluoride for 5 minutes.

Step 5. The small portion of the premix/fluoride blend, step 4, was then blended into the bulk of the premix for 5 minutes.

Step 6 The lubricant, talc and magnesium stearate, were added to the formulation, step 5, and blended for 3 minutes.

Step 7. The directly compressible blend, step 6, was tableted with 15.875 mm, beveled edge, standard concave tooling into chewable tablets with the following parameters:
(i) Tablet weight: The weight was ±5% of the total tablet weight as indicated on the formulation examples. (SEE formulations 1 through 17 (Lots 1 through 17) below.
(ii) Hardness: 3 Kp. Range 2-5 Kp.
(iii) Friability: NMT 10% Thickness: Record

### EXAMPLE 1 (LOT 1)

| Item No. | Material | Amt/tab | | mg/tab | % weight |
|---|---|---|---|---|---|
| 1 | Vitamin A (as Palmitate) | 2500 | IU | 1.38 | 0.173 |
| 2 | Vitamin D3 (as Cholecalciferol) | 400 | IU | 0.01 | 0.001 |
| 3 | Vitamin E (as Acetate) | 15 | IU | 10.05 | 1.256 |
| 4 | Folic Acid | 0.3 | mg | 0.3 | 0.038 |
| 5 | Niacin (as Niacinamide) | 13.5 | mg | 13.5 | 1.688 |
| 6 | Vitamin B1 (as Thiamine Mononitrate) | 1.05 | mg | 1.05 | 0.131 |
| 7 | Vitamin B12 (as Cyanocobalmin) | 4.5 | mcg | 0.0045 | 0.001 |
| 8 | Vitamin B2 (Riboflavin) | 1.2 | mg | 1.2 | 0.15 |
| 9 | Vitamin B6 (as \grave{ }Pyroxidine HCL) | 1.05 | mg | 1.05 | 0.131 |
| 10 | Vitamin C (as Ascorbic Acid) | 60 | mg | 60 | 7.5 |
| 11 | Microcrystalline Cellulose | 160 | mg | 160 | 20.000 |
| 12 | Sugar, compressible | 415 | mg | 415 | 51.875 |
| 13 | Xylitol | 136.40 | mg | 136.40 | 17.05 |

| | | |
|---|---|---|
| Total: | 800 | 100.000 |

The formulation of Example 1 was determined to be poor, gritty and bitter tasting.

### EXAMPLE 2 (LOT 2)

| Item No. | Material | Amt/tab | | mg/tab | % weight |
|---|---|---|---|---|---|
| 1 | Vitamin A (as Palmitate) | 2500 | IU | 1.38 | 0.095 |
| 2 | Vitamin C (as Ascorbic Acid) | 60 | mg | 60 | 7.576 |
| 3 | Vitamin D3 (as Cholecalciferol) | 400 | IU | 0.01 | 0.001 |
| 4 | Vitamin E (as Acetate) | 15 | IU | 10.05 | 1.269 |
| 5 | Vitamin B1 (as Thiamine Mononitrate) | 1.05 | mg | 1.05 | 0.133 |
| 6 | Vitamin B2 (Riboflavin) | 1.2 | mg | 1.2 | 0.152 |
| 7 | Niacin (as Niacinamide) | 13.5 | mg | 13.5 | 1.705 |
| 8 | Vitamin B6 (as \grave{ }Pyroxidine HCL) | 1.05 | mg | 1.05 | 0.133 |
| 9 | Folate | 0.3 | mg | 0.3 | 0.038 |
| 10 | Vitamin B12 (as Cyanocobalmin) | 4.5 | mcg | 0.0045 | 0.0006 |
| 11 | Citric Acid | 5 | mg | 5 | 0.631 |
| 12 | Malic Acid | 10 | mg | 10 | 1.263 |
| 13 | Microcrystalline Cellulose | 160 | mg | 160 | 20.00 |
| 14 | Sucralose | 1 | mg | 1 | 0.126 |
| 15 | Xylitol | 534.41 | mg | 534.41 | 66.80 |

| | | |
|---|---|---|
| Total: | 800 | 100.000 |

The formulation of Example 2 was determined to be a good tasting formulation. The formulation was improved over lot 1 when sucrose was replaced with sucralose. The decrease in bulk weight from 415 mg of compressible sugar allows a four fold increase in xylitol to 67% tablet weight. The malic and citric acids imparted a slight taste to the formulation.

### EXAMPLE 3 (LOT 2 A)

| Item No. | Material | Amt/tab | | mg/tab | % weight |
|---|---|---|---|---|---|
| 1 | Vitamin A (as Palmitate) | 2500 | IU | 1.38 | 0.159 |
| 2 | Vitamin C (as Ascorbic Acid) | 60 | mg | 60 | 6.956 |
| 3 | Vitamin D3 (as Cholecalciferol) | 400 | IU | 0.01 | 0.001 |
| 4 | Vitamin E (as Acetate) | 15 | IU | 10.05 | 1.165 |
| 5 | Vitamin B1 (as Thiamine Mononitrate) | 1.05 | mg | 1.05 | 0.121 |
| 6 | Vitamin B2 (Riboflavin) | 1.2 | mg | 1.2 | 0.139 |
| 7 | Niacin (as Niacinamide) | 13.5 | mg | 13.5 | 1.565 |
| 8 | Vitamin B6 (as \grave{ }Pyroxidine HCL) | 1.05 | mg | 1.05 | 0.121 |
| 9 | Folate | 0.3 | mg | 0.3 | 0.034 |
| 10 | Vitamin B12 (as Cyanocobalmin) | 4.5 | mcg | 0.0045 | 0.0005 |
| 11 | Citric Acid | 5 | mg | 5 | 0.579 |
| 12 | Malic Acid | 10 | mg | 10 | 1.159 |
| 13 | Microcrystalline Cellulose | 160 | mg | 160 | 18.550 |
| 14 | Sucralose | 1 | mg | 1 | 0.115 |
| 15 | Xylitol | 534.41 | mg | 534.41 | 61.969 |
| 16 | Ferrous Fumerate* (elemental iron) | 62.52 | | 62.52 | 7.248 |

| | | |
|---|---|---|
| Total: | 862.52 | 100.000 |

*equivalent to 12 mg elemental iron

The formulation of Example 3 is a replicate of lot 2, a combination of vitamins and elemental iron. The iron imparted a slight metallic after-taste to the product but overall the taste was very much acceptable.

### EXAMPLE 4 (LOT 5)

| Item No. | Material | Amt/tab | | mg/tab | % weight |
|---|---|---|---|---|---|
| 1 | Vitamin A (as Palmitate) | 2500 | IU | 1.38 | 0.173 |
| 2 | Vitamin C (as Ascorbic Acid) | 60 | mg | 60 | 7.5 |
| 3 | Vitamin D3 (as Cholecalciferol) | 400 | IU | 0.01 | 0.001 |
| 4 | Vitamin E (as Acetate) | 15 | IU | 10.05 | 1.256 |
| 5 | Vitamin B1 (as Thiamine Mononitrate) | 1.05 | mg | 1.05 | 0.131 |
| 6 | Vitamin B2 (Riboflavin) | 1.2 | mg | 1.2 | 0.15 |
| 7 | Niacin (as Niacinamide) | 13.5 | mg | 13.5 | 1.687 |
| 8 | Vitamin B6 (as \grave{ }Pyroxidine HCL) | 1.05 | mg | 1.05 | 0.131 |
| 9 | Folate | 0.3 | mg | 0.3 | 0.037 |
| 10 | Vitamin B12 (as Cyanocobalmin) | 4.5 | mcg | 0.0045 | 0.0006 |
| 11 | Citric Acid | 5 | mg | 5 | 0.625 |
| 12 | Malic Acid | 10 | mg | 10 | 0.012 |
| 13 | Microcrystalline Cellulose | 160 | mg | 160 | 20.00 |
| 14 | Sucralose | 1 | mg | 1 | 0.125 |
| 15 | Xylitol | 525.69 | mg | 525.69 | 65.711 |
| 16 | FD & C Yellow #5 Lake | 1.6 | mg | 1.6 | 0.20 |
| 17 | FD & C Yellow #6 Lake | 4.4 | mg | 4.4 | 0.55 |
| 18 | FD & C Red #40 Aluminum Lake | 2 | mg | 2 | 0.25 |
| | 35-42% | | | | |
| 19 | Orange Flavor | 2.4 | mg | 2.4 | 0.3 |

| | | |
|---|---|---|
| Total: | 800 | 100.000 |

The formulation of Example 4 was determined to be a very good tasting product. The taste was enhanced by the orange flavor.

### EXAMPLE 5 (LOT 14)

| Item No. | Material | Amt/tab | | mg/tab | % weight |
|---|---|---|---|---|---|
| 1 | Vitamin A (as Palmitate) | 2500 | IU | 1.38 | 0.151 |
| 2 | Vitamin C (as Ascorbic Acid) | 60 | mg | 60 | 6.593 |
| 3 | Vitamin D3 (as Cholecalciferol) | 400 | IU | 0.01 | 0.0012 |
| 4 | Vitamin E (as Acetate) | 15 | IU | 10.05 | 1.104 |
| 5 | Vitamin B1 (as Thiamine Mononitrate) | 1.05 | mg | 1.05 | 0.115 |
| 6 | Vitamin B2 (Riboflavin) | 1.2 | mg | 1.2 | 0.131 |
| 7 | Niacin (as Niacinamide) | 13.5 | mg | 13.5 | 1.483 |
| 8 | Vitamin B6 (as \grave{ }Pyroxidine HCL) | 1.05 | mg | 1.05 | 0.115 |
| 9 | Folate | 0.3 | mg | 0.3 | 0.032 |
| 10 | Vitamin B12 (as Cyanocobalmin) | 4.5 | mcg | 0.0045 | 0.0004 |
| 11 | Magnesium (as Magnesium | 30 | mg | * | 3.296 |
| | glycerophosphate, anhydrous) | | | | |
| 12 | Phosphorous (as Magnesium | 38 | mg | * | 4.175 |
| | glycerophosphate, anhydrous) | | | | |
| 13 | Copper (as Cupric Oxide) | 2 | mg | 2 | 0.219 |
| 14 | Citric Acid | 7.6 | mg | 7.6 | 0.835 |
| 15 | FD & C Red #40 | 8 | mg | 8 | 0.879 |
| 16 | Malic Acid | 12 | mg | 12 | 1.318 |
| 17 | Microcrystalline Cellulose | 160 | mg | 160 | 17.582 |
| 18 | Sucralose | 2.6 | mg | 2.6 | 0.285 |
| 19 | Xylitol | 377.66 | mg | 377.66 | 41.50 |
| 20 | Strawberry Flavor | 2.4 | mg | 2.4 | 0.3 |

| | | |
|---|---|---|
| Total: | 910 | 100.000 |

* Magnesium and phosphorous are derived from 242.30 mg of magnesium glycerophosphate (26.63% of 910 mg total tablet weight)

### EXAMPLE 6 (LOT 15)

| Item No. | Material | Amt/tab | | mg/tab | % weight |
|---|---|---|---|---|---|
| 1 | Vitamin A (as Palmitate) | 2500 | IU | 1.38 | 0.1531 |
| 2 | Vitamin C (as Ascorbic Acid) | 60 | mg | 60 | 6.66 |
| 3 | Vitamin D3 (as Cholecalciferol) | 400 | IU | 0.01 | 0.0011 |
| 4 | Vitamin E (as Acetate) | 15 | IU | 10.05 | 1.1164 |
| 5 | Vitamin B1 (as Thiamine Mononitrate) | 1.05 | mg | 1.05 | 0.116 |
| 6 | Vitamin B2 (Riboflavin) | 1.2 | mg | 1.2 | 0.133 |
| 7 | Niacin (as Niacinamide) | 13.5 | mg | 13.5 | 1.50 |
| 8 | Vitamin B6 (as \grave{ }Pyroxidine HCL) | 1.05 | mg | 1.05 | 0.116 |
| 9 | Folate | 0.3 | mg | 0.3 | 0.033 |
| 10 | Vitamin B12 (as Cyanocobalmin) | 4.5 | mcg | 0.0045 | 0.0005 |
| 11 | Magnesium (as Magnesium | 30 | mg | * | 3.333 |
| | glycerophosphate, anhydrous) | | | | |
| 12 | Phosphorous (as Magnesium | 38 | mg | * | 4.222 |
| | glycerophosphate, anhydrous) | | | | |
| 13 | Copper (as Cupric Oxide) | 2 | mg | 2 | 0.219 |
| 14 | Citric Acid | 7.6 | mg | 7.6 | 0.844 |
| 15 | Malic Acid | 12 | mg | 12 | 1.333 |
| 16 | Cappuccino Flavor | 8 | mg | 8 | 0.888 |
| 17 | Microcrystalline Cellulose | 160 | mg | 160 | 17.777 |
| 18 | Sucralose | 2.6 | mg | 2.6 | 0.288 |
| 19 | Xylitol | 376.96 | mg | 377.96 | 41.88 |

| | | |
|---|---|---|
| Total: | 900 | 100.000 |

* Magnesium and phosphorous are derived from 242.30 mg of magnesium glycerophosphate (26.92% of 900 mg total tablet weight)

The formulations of Examples 5 and 6 incorporated minerals with the vitamins in the formulation base of xylitol, sucralose and microcrystalline cellulose. Both formulations were very tasty and acceptable.

### EXAMPLE 7 (LOT 17)

| Item No. | Material | Amt/tab | | mg/tab | % weight |
|---|---|---|---|---|---|
| 1 | Vitamin A (as Palmitate) | 2500 | IU | 1.38 | 0.095 |
| 2 | Vitamin C (as Ascorbic Acid) | 60 | mg | 60 | 7.576 |
| 3 | Vitamin D3 (as Cholecalciferol) | 400 | IU | 0.01 | 0.001 |
| 4 | Vitamin E (as Acetate) | 15 | IU | 10.05 | 1.269 |
| 5 | Vitamin B1 (as Thiamine Mononitrate) | 1.05 | mg | 1.05 | 0.133 |
| 6 | Vitamin B2 (Riboflavin) | 1.2 | mg | 1.2 | 0.152 |
| 7 | Niacin (as Niacinamide) | 13.5 | mg | 13.5 | 1.705 |
| 8 | Vitamin B6 (as \grave{ }Pyroxidine HCL) | 1.05 | mg | 1.05 | 0.133 |
| 9 | Folate | 0.3 | mg | 0.3 | 0.038 |
| 10 | Vitamin B12 (as Cyanocobalmin) | 4.5 | mcg | 0.0045 | 0.0006 |
| 11 | Citric Acid | 5 | mg | 5 | 0.631 |
| 12 | Malic Acid | 10 | mg | 10 | 1.263 |
| 13 | Orange Flavor | 2.4 | mg | 2.4 | 0.303 |
| 14 | Microcrystalline Cellulose | 160 | mg | 160 | 20.202 |
| 15 | Sucralose | 1 | mg | 1 | 0.126 |
| 16 | Xylitol | 517.13 | mg | 517.13 | 65.294 |
| 17 | Sodium Fluoride | 0.552 | mg | 0.552 | 0.070 |
| 18 | Talc | 4.00 | mg | 4 | 0.505 |
| 19 | Magnesium Stearate | 4.00 | mg | 4 | 0.505 |

| | | |
|---|---|---|
| Total: | 792 | 100.000 |

The formulation of Example 7 lot 17 incorporates vitamins, minerals and the active nutrient sodium fluoride. The formulation was tasty with good palatability.

As shown in the above examples, the combination of xylitol, sucralose and microcrystalline cellulose as an excipient base significantly improved the taste and palatability of the chewable pharmaceutical and nutritional supplement tablets. By carefully selecting these ingredients and the percentile range of these ingredients, it is possible to prepare chewable pharmaceutical and nutritional supplement tablets that have improved taste, mouthfeel, stability, and greater likelihood for dosage compliance and use, particularly among children, as compared to formulations currently in use. Further, by carefully selecting these ingredients and by carefully selecting the percentile range of these ingredients, it is possible to prepare chewable pharmaceutical and nutritional supplement tablets that are suitable for administration to persons and animals with diabetes and hypoglycemia, and do not promote tooth decay or dental caries.

While the present invention has been described with reference to one or more embodiments set forth in considerable detail for the purposes of making a complete disclosure of the invention, such embodiments are merely exemplary, and are not intended to limit or represent an exhaustive enumeration of all aspects of the invention. The scope of the invention, therefore, shall be defined solely by the following claims. Further, it will be apparent to those of skill in the art that numerous changes may be made in such details without departing from the spirit and the principles of the invention.

We claim:

1. An excipient base for a chewable tablet, the excipient base comprising xylitol, sucralose and microcrystalline cellulose.

2. The excipient base of claim 1, wherein the xylitol is between 40-70% of the total tablet weight, the microcrystalline cellulose is between 15-20% of the total tablet weight, and the sucralose is between 0.1-0.2% of the total tablet weight.

3. The excipient base of claim 2, wherein the microcrystalline cellulose is 20% of the total tablet weight and the sucralose is 0.2% of the total tablet weight.

4. A chewable tablet of claim 1 further comprising one or more active ingredients selected from the group consisting of pharmaceutical active drugs, vitamins, minerals, nutrients, dietary supplements and food ingredients.

5. The chewable tablet of claim 4 wherein and the xylitol is between 40-70% of the total tablet weight, the microcrystalline cellulose is between 15-20% of the total tablet weight, and the sucralose is between 0.1-0.2% of the total tablet weight.

6. The chewable tablet of claim 4 wherein the one or more vitamins are vitamin A, C, D3, E, B1, B2, Niacin, B6, Folate or B12, or any combination thereof.

7. The chewable tablet of claim 4 wherein the one or more minerals are magnesium, phosphorous, copper, sodium fluoride or iron, or any combination thereof.

8. The chewable tablet of claim 4 wherein the one or more minerals include magnesium, phosphorous, copper, zinc, selenium, biotin, iodine, calcium, zinc, chromium, molybdenum, pantothenic acid, manganese or iron.

9. The chewable tablet of claim 8 wherein the magnesium is between 5 to 6% of the total weight of the tablet, the phosphorous is between 2 to 6% of the weight of the tablet, and the copper is between 2 to 3% of the weight of the tablet.

10. The chewable tablet of claim 9 wherein the copper is less than two milligrams.

11. The chewable tablet of claim 4 wherein the active nutrients is protein, carbohydrate and/or lipid.

12. The chewable tablet of claim 4 wherein the food ingredient is an amino acid.

13. The chewable tablet of claim 4 wherein the pharmaceutical active ingredient is an analgesics, antacids or laxatives.

14. The chewable tablet of claim 4 wherein the dietary supplement is fluoride and/or elemental iron.

15. The chewable tablet of claim 14 wherein the fluoride is less than two milligrams.

16. The chewable tablet of claim 8 wherein the one or more minerals comprise no more than 9% by weight of the total tablet, and wherein the tablet further comprises (a) one or more vitamins, (b) one or more active nutrients or food ingredients, (c) a pharmaceutical active ingredient, or (d) one or more dietary supplements, and wherein (a)-(d) individually or in combination amount to 0.07-15% of the total weight of the tablet.
